(19) United States
(12) Patent Application Publication
(10) Pub. No.: US 2021/0177057 A1
(43) Pub. Date: Jun. 17, 2021

(54) ELECTRONIC CIGARETTE

(71) Applicant: Tuanfang LIU, Shenzhen (CN)

(72) Inventor: Tuanfang LIU, Shenzhen (CN)

(21) Appl. No.: 17/020,781

(22) Filed: Sep. 14, 2020

(30) Foreign Application Priority Data
Dec. 12, 2019 (CN) 201911273077.5
Dec. 12, 2019 (CN) 201922228546.3

(51) Int. Cl. A24F 40/46

(52) U.S. Cl. CPC A24F 40/46

(57) ABSTRACT

An electronic cigarette, including an atomization assembly and a battery assembly. The atomization assembly includes an atomizer and an e-liquid tank. The atomizer includes two or more heating elements. The e-liquid tank includes a plurality of compartments corresponding to the heating elements in number. The two or more heating elements are disposed in the plurality of compartments, respectively.

### CROSS-REFERENCE TO RELATED APPLICATIONS

Pursuant to 35 U.S.C. § 119 and the Paris Convention Treaty, this application claims foreign priority to Chinese Patent Application No. 201911273077.5 filed Dec. 12, 2019, and to Chinese Patent Application No. 201922228546.3 filed Dec. 12, 2019. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to an atomization assembly and an electronic cigarette comprising the same.

A conventional electronic cigarette includes only one atomizer equipped with only one heating element.

### SUMMARY

The disclosure also provides an electronic cigarette, comprising an atomization assembly and a battery assembly; the atomization assembly comprises an atomizer and an e-liquid tank; the atomizer comprises two or more heating elements; the e-liquid tank comprises a plurality of compartments corresponding to the heating elements in number; and the two or more heating elements are disposed in the plurality of compartments, respectively.

The battery assembly is disposed on the bottom end of the atomization assembly. The battery assembly comprises a bottom casing. The bottom casing is rotatable with respect to the battery assembly so as to switch the working mode of the atomizer.

The battery assembly comprises the top part and an electrode pair disposed on the top part. The atomization assembly comprises the bottom part and a joint disposed on the bottom part. The electrode pair is in contact with the joint for electric conduction. The bottom casing of the battery assembly is rotatable thus driving a switching printed circuit board (PCB) disposed on the top end of a rotating shaft to rotate to power on or power off the electrode pair, so as to switch the working mode of the atomizer.

The bottom casing is rotatable with respect to the battery assembly so as to switch the working mode of the atomizer to control the two or more heating elements disposed in the plurality of compartments of the e-liquid tank to work alone or simultaneously.

The atomization assembly further comprises a mouthpiece, a seal plug, a rubber gasket, a bottom cover, and a magnet. The atomizer is disposed on the rubber gasket. The rubber gasket is disposed on the bottom cover. The joint is disposed on the bottom end of the bottom cover to fix the atomizer. The bottom cover is disposed on a bottom part of the e-liquid tank to seal the e-liquid tank. The seal plug is disposed on the top part of the e-liquid tank to seal an e-liquid injection hole of the e-liquid tank; and the mouthpiece is disposed on the top end of the e-liquid tank.

The battery assembly further comprises a rotatable fixed seat, an insulation tube, an LED tube, a bolt, a decorative board, a battery, a control panel, a rubber ring, a support, and a battery case. The electrode pair is disposed on the support to supply power to the atomization assembly. The rubber ring is disposed on the support to insulate the electrode pair. The insulation tube is disposed on the top end of the rotating shaft. The rotatable fixed seat is disposed on the insulation tube to fix the rotating shaft. The switching PCB is disposed on the rotatable fixed seat and is connected to the rotating shaft. The LED tube is disposed on the bottom end of the rotating shaft. The rotating shaft is disposed in the support. The battery is soldered on the control panel and the control panel is fixed on the support. The support is disposed in the battery case. The bottom casing is disposed on the bottom end of the battery case, in contact with the LED tube to connect to and control the rotating shaft. The bolt is in threaded connection to the bottom end of the bottom casing. The LED tube is fixed in the bottom casing. The decorative board is attached to a bottom surface of the bottom casing; and the magnet is disposed on the top end of the support.

The atomization assembly comprises an atomizer and an e-liquid tank. The atomizer comprises two or more heating elements. The e-liquid tank comprises a plurality of compartments corresponding to the heating elements in number; and the two or more heating elements are disposed in the plurality of compartments, respectively. The plurality of compartments is filled with different e-liquid materials. The users can switch the working mode of the atomizer to vaporize different e-liquid materials.

### BRIEF DESCRIPTION OF THE DRAWINGS

FIG. 1 is an exploded view of an electronic cigarette according to one embodiment of the disclosure;

FIG. 2 is an exploded view of an atomization assembly of an electronic cigarette according to one embodiment of the disclosure;

FIG. 3 is an exploded view of a battery assembly of an electronic cigarette according to one embodiment of the disclosure;

FIG. 4 is a schematic diagram of an electronic cigarette according to one embodiment of the disclosure; and

FIG. 5 is a sectional view of an electronic cigarette according to one embodiment of the disclosure.

### DETAILED DESCRIPTION

To further illustrate, embodiments detailing an electronic cigarette are described below. It should be noted that the following embodiments are intended to describe and not to limit the disclosure.

In the disclosure, smoke materials refer to smoke oil, tobacco, tobacco and other materials used to produce smoke.

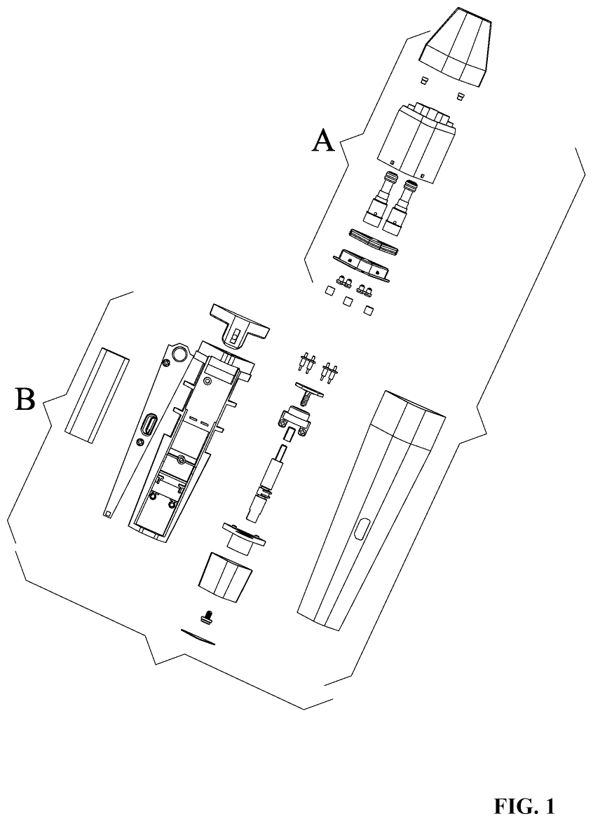

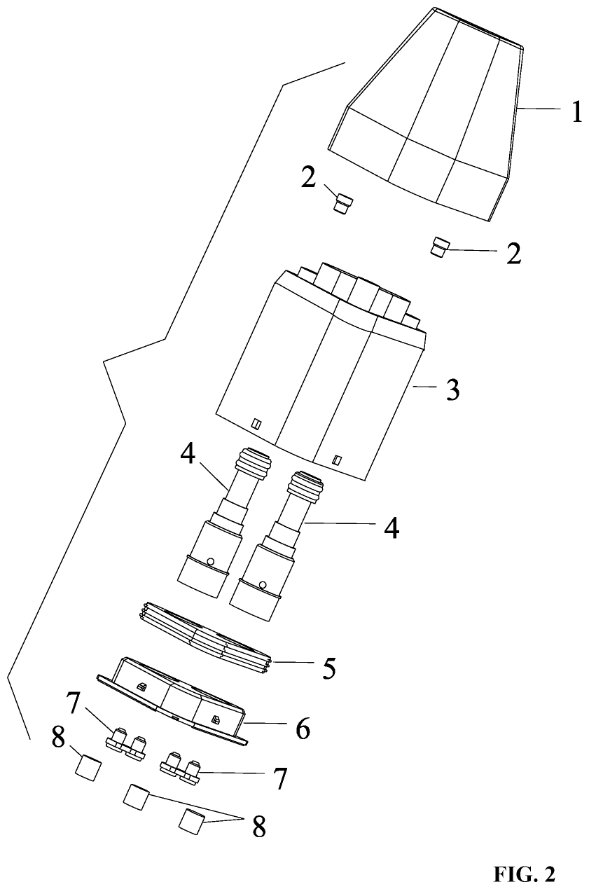

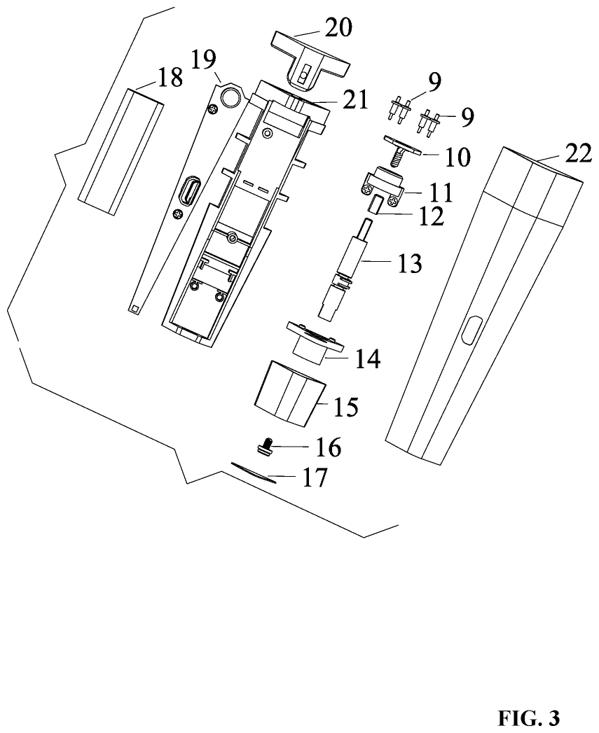

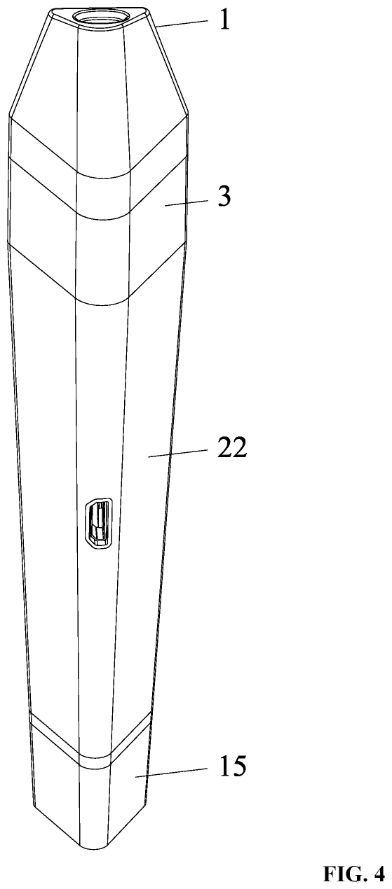

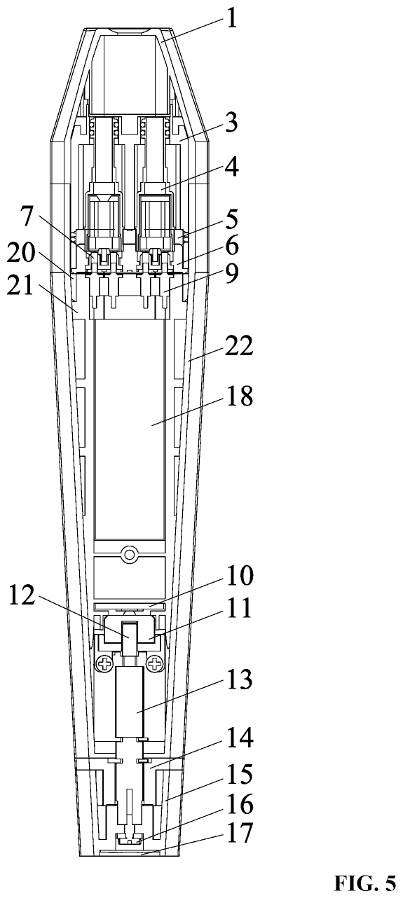

As shown in FIGS. 1-5, an electronic cigarette comprises two heating elements and an e-liquid tank 3 comprising two compartments. Specifically, the electronic cigarette comprises an atomization assembly A and a battery assembly B. The atomization assembly A a mouthpiece 1, a seal plug 2, an e-liquid tank 3, an atomizer 4, a rubber gasket 5, a bottom cover 6, a joint 7, and a magnet 8. The atomizer 4 is disposed on the rubber gasket 5. The rubber gasket 5 is disposed on the bottom cover 6. The joint 7 is disposed on the bottom end of the bottom cover 6 to fix the atomizer 4. The bottom cover 6 is disposed on a bottom part of the e-liquid tank 3 to seal the e-liquid tank. The seal plug 2 is disposed on the top part of the e-liquid tank 3 to seal an e-liquid injection hole of the e-liquid tank; and the mouthpiece 1 is disposed on the top end of the e-liquid tank 3.

The battery assembly B further comprises an electrode pair 9, a switching PCB 10, a rotatable fixed seat 11, an insulation tube 12, a rotating shaft 13, an LED tube 14, a bottom casing 15, a bolt 16, a decorative board 17, a battery 18, a control panel 19, a rubber ring 20, a support 21, and a battery case 22. The electrode pair 9 is disposed on the support 21 to supply power to the atomization assembly. The rubber ring 20 is disposed on the support 21 to insulate the electrode pair 9. The insulation tube 12 is disposed on the top end of the rotating shaft 13. The rotatable fixed seat 11 is disposed on the insulation tube 12 to fix the rotating shaft 13. The switching PCB 10 is disposed on the rotatable fixed seat 11 and is connected to the rotating shaft 13. The LED tube 14 is disposed on the bottom end of the rotating shaft 13. The rotating shaft 13 is disposed in the support 21. The battery 18 is soldered on the control panel 19 and the control panel 19 is fixed on the support 21. The support 21 is disposed in the battery case 22. The bottom casing 15 is disposed on the bottom end of the battery case 22, in contact with the LED tube 14 to connect to and control the rotating shaft 13. The bolt 16 is in threaded connection to the bottom end of the bottom casing 15. The LED tube 14 is fixed in the bottom casing 15. The decorative board 17 is attached to a bottom surface of the bottom casing 15; and the magnet 8 is disposed on the top end of the support 21. The atomization assembly A is disposed on the battery assembly B and is connected to the battery assembly B through the magnet 8.

The electronic cigarette comprises two heating elements and an e-liquid tank 3 comprising two compartments. The bottom casing 15 is rotatable with respect to the battery assembly B so as to switch the working mode of the atomizer to control the two heating elements respectively disposed in the two compartments of the e-liquid tank to work alone or simultaneously. The two compartments are filled with different e-liquid materials. The users can switch the working mode of the atomizer to vaporize different e-liquid materials.

It will be obvious to those skilled in the art that changes and modifications may be made, and therefore, the aim in the appended claims is to cover all such changes and modifications.

What is claimed is:

1. An electronic cigarette, comprising an atomization assembly and a battery assembly; wherein the atomization assembly comprises an atomizer and an e-liquid tank; the atomizer comprises two or more heating elements; the e-liquid tank comprises a plurality of compartments corresponding to the heating elements in number; and the two or more heating elements are disposed in the plurality of compartments, respectively.

2. The electronic cigarette of claim 1, wherein the battery assembly is disposed on a bottom end of the atomization assembly; the battery assembly comprises a bottom casing; the bottom casing is rotatable with respect to the battery assembly so as to switch a working mode of the atomizer.

3. The electronic cigarette of claim 2, wherein the battery assembly comprises a top part and an electrode pair disposed on the top part; the atomization assembly comprises a bottom part and a joint disposed on the bottom part; the electrode pair is in contact with the joint for electric conduction; the bottom casing of the battery assembly is rotatable thus driving a switching PCB disposed on a top end of a rotating shaft to rotate to power on or power off the electrode pair, so as to switch the working mode of the atomizer.

4. The electronic cigarette of claim 2, wherein the bottom casing is rotatable with respect to the battery assembly so as to switch the working mode of the atomizer to control the two or more heating elements disposed in the plurality of compartments of the e-liquid tank to work alone or simultaneously.

5. The electronic cigarette of claim 3, wherein the bottom casing is rotatable with respect to the battery assembly so as to switch the working mode of the atomizer to control the two or more heating elements disposed in the plurality of compartments of the e-liquid tank to work alone or simultaneously.

6. The electronic cigarette of claim 3, wherein the atomization assembly further comprises a mouthpiece, a seal plug, a rubber gasket, a bottom cover, and a magnet; the atomizer is disposed on the rubber gasket; the rubber gasket is disposed on the bottom cover; the joint is disposed on a bottom end of the bottom cover to fix the atomizer; the bottom cover is disposed on a bottom part of the e-liquid tank to seal the e-liquid tank; the seal plug is disposed on a top part of the e-liquid tank to seal an e-liquid injection hole of the e-liquid tank; and the mouthpiece is disposed on a top end of the e-liquid tank.

7. The electronic cigarette of claim 3, wherein the battery assembly further comprises a rotatable fixed seat, an insulation tube, an LED tube, a bolt, a decorative board, a battery, a control panel, a rubber ring, a support, and a battery case; the electrode pair is disposed on the support to supply power to the atomization assembly; the rubber ring is disposed on the support to insulate the electrode pair; the insulation tube is disposed on the top end of the rotating shaft; the rotatable fixed seat is disposed on the insulation tube to fix the rotating shaft; the switching PCB is disposed on the rotatable fixed seat and is connected to the rotating shaft; the LED tube is disposed on a bottom end of the rotating shaft; the rotating shaft is disposed in the support; the battery is soldered on the control panel and the control panel is fixed on the support; the support is disposed in the battery case; the bottom casing is disposed on a bottom end of the battery case, in contact with the LED tube to connect to and control the rotating shaft; the bolt is in threaded connection to a bottom end of the bottom casing; the LED tube is fixed in the bottom casing; the decorative board is attached to a bottom surface of the bottom casing; and the magnet is disposed on a top end of the support.
